(19) United States
(12) Patent Application Publication
(10) Pub. No.: US 2005/0202566 A1
(43) Pub. Date: Sep. 15, 2005

(54) Device and method for determining parameters

(76) Inventor: Maurice Frojmovic, Quebec (CA)

Correspondence Address: BACHMAN & LAPOINTE, P.C., 900 CHAPEL STREET, SUITE 1201, NEW HAVEN, CT, 06510, US

(21) Appl. No.: 10/504,181

(22) PCT Filed: Feb. 13, 2003
(86) PCT No.: PCT/CA03/00214

§ 371 Date: Mar. 3, 2005

Related U.S. Application Data
(60) Provisional application No. 60/356,166, filed on Feb. 14, 2002.

(51) Int. Cl. G01N 33/48

(52) U.S. Cl. 436/63; 422/73

(57) ABSTRACT

A method and apparatus for determining a parameter of polymers, particles or cells over a wide range of flow conditions, more especially for cells in an animal body fluid, especially a cell aggregation parameter; a sample is subjected in a chamber (18, 62) to flow and shear conditions which mimic conditions of the particles or cells in their normal environment; the sample is exposed, under the flow and shear conditions in the chamber (18, 62), to a polymer, particle or cell parameter determining operation.

### BACKGROUND OF THE INVENTION

1. Field of the Invention

This invention relates to a method and apparatus for determining a parameter of polymers, particles or cells in a liquid vehicle, especially cells in an animal body fluid.

2. Description of Prior Art

There is a growing awareness that drug efficacy for inhibition of platelet aggregation can vary with flow conditions, which can be controlled in microflow devices normally studied at room temperature Turner et al., (Blood, 98; 3340-45, 2001), reported that shear induced aggregation of platelets spontaneously occurred in isolated platelets suspensions only at extremely high shear rates (10,000 s⁻¹), but readily occurred for whole blood suspensions at normal physiological arterial shear rates, and increasingly with shear (from 750-3000 s⁻¹). Much greater inhibition of platelet aggregation was seen in whole blood at the lower shear (750-100 s⁻¹) than at higher shear (3000 s⁻¹)(>3-fold more inhibition), when evaluating either an anti-ADP receptor drug (AstraZeneca product ARMX) or an anti-GPIIbIIIa receptor drug (ReoPro).

Hanson et al., (American Heart Journal, 135 (5); S132-45, 1998) report the need to evaluate the efficacy of antithrombotic drugs as a function of varied flow conditions. They specifically point to the ability of aspirin and Linotroban, a thromboxane A2 antagonist, to increasingly inhibit thrombus formation in ex vivo flow models using whole blood, with increasing shear. Hanson et al., point to similar effects with inhibitors of von Willebrand factor platelet interactions, this was also discussed by the studies of Frojmovic et al reported in the same Journal Supplement (American Heart Journal: 135 (5); S 119-31, 1998).

In reality a body fluid is subject to a wide range of flow conditions in the body. In the case of blood, for example, the location of the blood in the circulatory system and pathological circumstances affect the flow and parameters such as adhesion and aggregation of cells, and shear to which the flowing blood is subjected in its circulatory travel.

Testing of parameters of body fluids is useful in diagnosis and in evaluating the benefits or usefulness of pharmacological agents. Such testing, when carried out in vitro typically involves the body fluid in a static state or under specific flow conditions. U.S. Pat. No. 6,043,871 by Solen et al., describes an instrument for measuring platelet aggregation in whole blood, using cylindrical plastic tubes under specific flow conditions, therefore without special control of patho-physiological flow rates and shear stresses, the instrument is limited to measuring only very large platelet aggregates.

Existing apparatus and methods perform testing on body fluids in a state, which is not representative of the state of the fluids in the body.

A couette is a device employed for determining viscosity of a liquid, and comprises two concentric cylinders with a small gap between the inner wall of the outer stationary cylinder and the outer wall of the rotatable inner cylinder. Liquid is introduced into the gap and the rotatable inner cylinder is rotated to develop shear in the liquid.

The resistance provided by the liquid to rotation of the inner cylinder provides a measure of viscosity of the liquid.

It has been proposed previously to study aggregation of human blood platelets as a function of shear rates varied between 100 and 1000 s⁻¹. (Frojmovic et al, Biophys. J. 66: 2190-2201, 1994).

It would be highly advantageous in diagnosis and in evaluation of efficacy of pharmacologic agents in body fluids, if body fluids could be tested under conditions simulating those in the body.

### SUMMARY OF THE INVENTION

It is an object of this invention to provide a method and apparatus for determining parameters of polymers, particles or cells in a flowing liquid vehicle, especially cells in animal body fluids.

It is a particular object of this invention to provide such a method and apparatus in which dynamic, real-time measurements can be carried out, or, in other words, in which a body fluid is evaluated under conditions representative of the conditions to which the fluid is exposed in the body.

In accordance with one embodiment of the invention there is provided a method of determining a parameter of polymers, particles or cells in a liquid vehicle comprising: a) introducing a sample of a liquid vehicle containing suspended particles or cells into a chamber, b) subjecting said sample, in said chamber, to flow and shear conditions which mimic conditions of such particles or cells in their normal environment, and c) exposing said sample, under said flow and shear conditions in said chamber, to at least one polymer, particle or cell parameter determining operation.

In particular the sample may comprise the polymers, particles or cells in a colloidal suspension.

In another embodiment of the invention there is provided an apparatus for determining a parameter of polymers, particles or cells in a liquid comprising: a) a housing defining a chamber for receiving a sample of the liquid vehicle containing suspended polymers, particles or cells; b) means for subjecting said sample, in said chamber, to flow and shear conditions which mimic conditions of such particles or cells in their normal environment, and c) means for exposing said sample, under said flow and shear conditions in said chamber, to at least one polymer, particle or cell parameter determining operation.

In particular embodiment of the invention there is provided an apparatus for determining a parameter of polymers, particles or cells in a liquid comprising: a housing, a rotatable inner cylinder in said housing, a stationary cylindrical wall spaced apart from and circumscribing said cylinder with an annular chamber defined between said wall and said rotatable inner cylinder, motor means for rotating said inner cylinder relative to said wall, and polymer, particle or cell parameter determining means mounted in said housing for operable communication with said annular chamber.

In another particular embodiment of the invention there is provided an apparatus for determining a parameter of polymers, particles or cells in a liquid comprising: a housing, a tube flow chamber in said housing, a variable liquid flow means, polymer, particle or cell parameter determining means mounted in said housing for operable communication with said tube flow chamber.

Other embodiments of the invention including important structures of apparatus will be apparent from the detailed description hereinafter.

### DESCRIPTION OF PREFERRED EMBODIMENTS

The invention is more particularly described by reference to the embodiments in which a parameter of cells in an animal body fluid are to be determined.

The invention is applicable to body fluids containing cells, generally, especially human body fluids. The invention is of particular interest in the investigation of parameters of cells involved in human diseases, for example, platelets and leukocytes in cardiovascular disease, and vascular, blood and metastatic cancer cells in oncology.

The body fluid will typically be subjected to the flow and shear conditions, in accordance with the invention, in the presence of a pharmacologic agent, for example, an activator or inhibitor believed to have an effect on a pathological condition associated with the cells of the fluid. In such case the invention assists in determining the efficacy or otherwise of the activator or inhibitor on the pathological condition.

In particular, an evaluation of adherence or aggregation of the cells in the fluid may be made, in the presence of the activator or inhibitor, while the fluid is under conditions mimicking the varying conditions in the body.

One evaluation involves transmitting a light beam from a source through the sample of the body fluid, to a detector, developing a signal responsive to the detected light at the detector and evaluating cell adherence and cell aggregation from said signal.

The signal may be in the form of a voltage output and changes in voltage are detected as a measure of turbidity of the cells in the sample. The turbidity is then a measure of large aggregate formation of cells.

The signal may also be in the form of a voltage output and changes in oscillation about the voltage are detected expressed as root mean square values, as a measure of onset of microaggregation.

In another embodiment, an intense light source at distinct wavelengths for exciting relevant chromophores within the flow device, and measuring light intensity at the relevant emission wavelength, is transmitted through the sample. This permits evaluations of biochemical events or molecular markers, such as secretion of growth factors, or expression of proteases or thrombin, dynamically in real time for sheared cell suspensions. Thus, using reporting chromogenic substrates for protease activities or fluorescently-labelled reports for a wide variety of cell functions, cell suspensions varying from isolated platelets or cancer cells to cell mixtures as complex and whole blood can be evaluated. In further embodiments, to flow cytometry (FCM); fluorescent labelling of cells or molecules will further allow particle analyses of molecular properties by FCM.

In further embodiments the sample or a portion of the sample may be withdrawn from the apparatus of the invention and subjected to flow cytometry. This use of flow cytometry is particularly preferred for mixed cell suspensions as complex as whole blood.

### BRIEF DESCRIPTION OF DRAWINGS

FIG. 1 is a schematic representation of apparatus of the invention with a plurality of units;

FIG. 2 is a schematic representation of a unit of the invention, in cross-section;

FIG. 3 is a detail of DPDA optical measurement employing the unit of FIG. 2;

FIG. 4 illustrates the relationship between cell aggregation and light transmission as measured by the DPDA method of the invention;

FIG. 5 is a schematic representation of variations in shear in blood flowing in an artery;

FIG. 6 is a schematic representation of a preferred embodiment of a tube flow chamber;

FIG. 7A is a schematic representation of a sample's course from flow chamber to flow cytometer;

FIG. 7B is a typical fluorescence scan generated with a fluorescence signal, in this case it is a FITC (Fluorescein Isothiocyanate Chromophore) vs the number of particles analysed; and

FIG. 7C is a typical scattering scan generated of FSC (Forward Scattering) vs SSC (Side Scattering) typically showing single and platelet aggregate numbers and sizes.

### DESCRIPTION WITH REFERENCE TO DRAWINGS

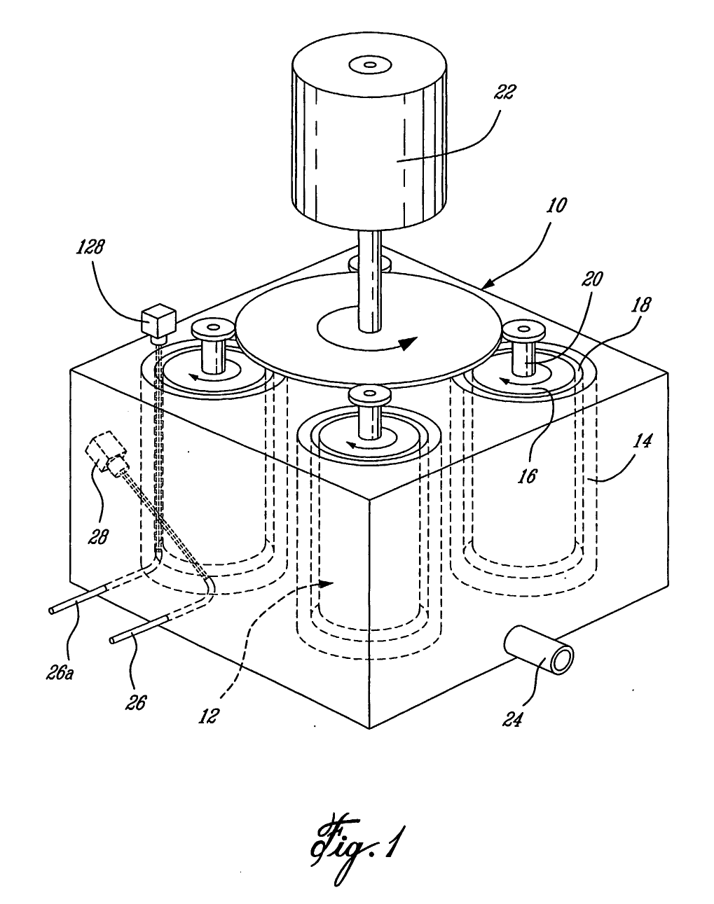

With further reference to FIG. 1, a schematic representation shows an assembly 10 which includes a plurality of units or cartridges, 12.

Each unit 12 comprises an outer stationary cylinder 14 and an inner rotatable cylinder 16. An annular chamber 18 is defined between outer stationary cylinder 14 and inner rotatable cylinder 16.

Each rotatable cylinder 16 has a spindle 20 for rotation therewith and the spindles 20 are driven by a common motor 22. The motor 22 can be replaced by a dedicated motor for each cylinder 16.

Each unit or couette 12 has a sampling port 24 which communicates with annular chamber 18. The couette 12 may be defined as a microcouette.

Each unit 12 has a light guide (or LED) 26 and detector 28, shown for transmitted light as in FIG. 3. There may be different entries and exits for the light.

An optional detector 128 measuring Transmitted Light (TA) using FSC light guide 26a may be employed in another option for dynamic focal point light scattering (DLS), with the light guide 26a placed at a different location from that shown for transmitted light to detector 28, rather entering below the rotatable cylinder 16 and passing through the entire suspension, leading to detector 128.

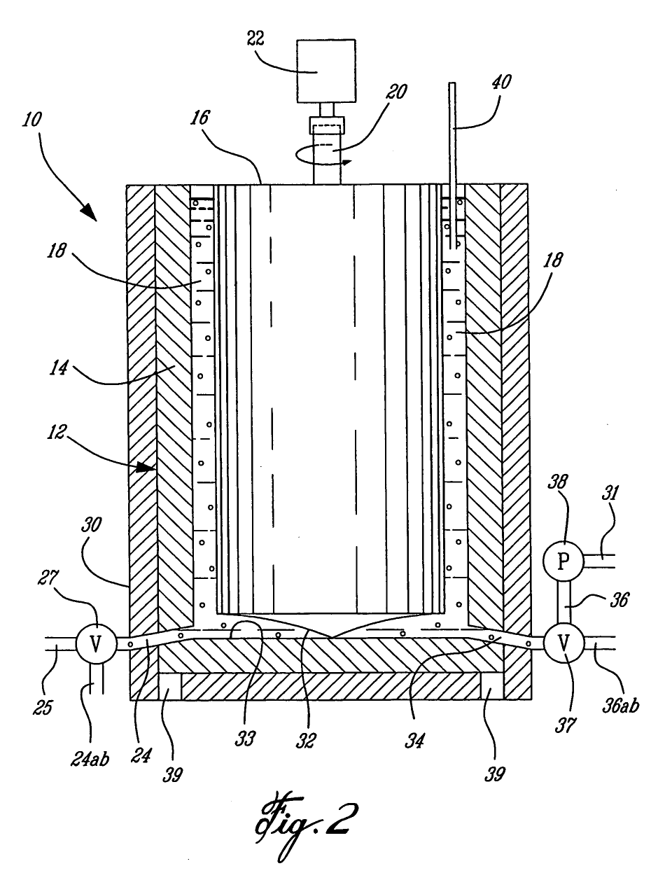

With further reference to FIG. 2, there is shown a detail of a unit 12 of FIG. 1.

Unit 12 includes housing 30 which releasably contains the combination of outer cylinder 14 and inner cylinder 16. Inner cylinder 16 has a conical end 32 supported on the floor 33 of outer cylinder 14.

An inlet 34 for introduction of a sample into annular chamber 18 extends through housing 30 and outer cylinder 14. A conduit 36 extends into inlet 34. A pump 38 is located in conduit 36 to pump the sample from a source into annular chamber 18, but not before passing through a 2-way or multiple way valve 37. Similarly in the outlet tube 25 of the chamber 18, there is a sampling chamber 24 and a 2-way or multiple way valve 27. The spring-loaded retraction/loading 39 of the couette allows ready insertion and removal of prefabricated, disposable inner and outer cylinder coupled units which are coupled to the motor drive.

A plunge needle 40 is provided to introduce additives into annular chamber 18.

With further reference to FIG. 3, there is shown a detail of a unit 12. An LED, 26, fed by a 50 mA DC source 40, provides a beam of light for passage through the sample in the annular space 18. The emerging light is received by detector 28 which provides a D.C. voltage output 42 which may be employed as described herein to provide a measure of turbidity of the sample; and an A.C. voltage output 44 to provide a root mean square signal as described herein which provides a measure of on-set of aggregation.

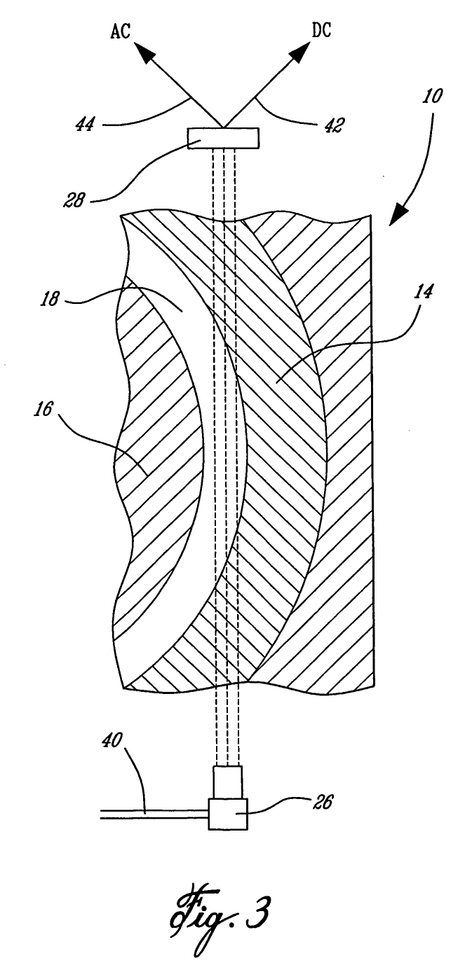

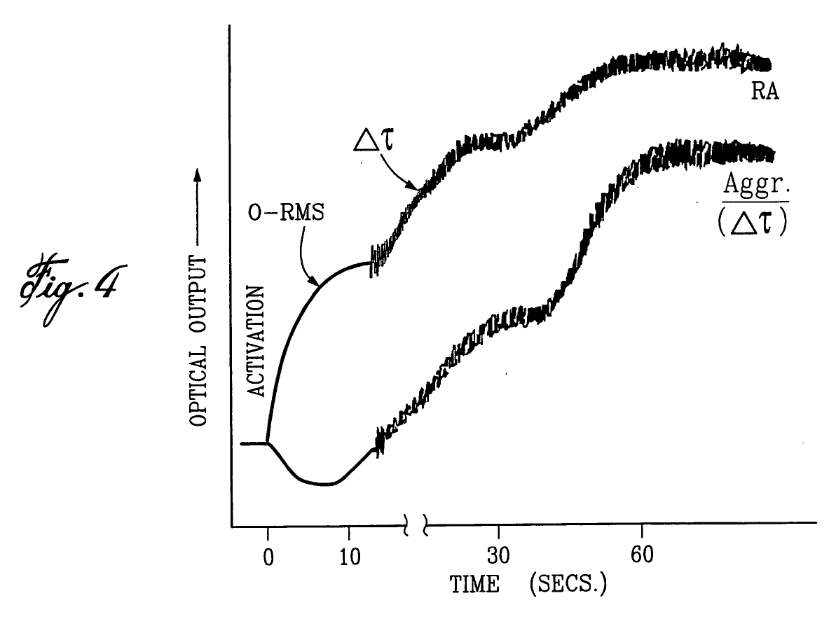

The relationship between aggregation and the root mean square value (O-RMS) is more particularly illustrated in FIG. 4. Schematic representative optical output tracings for changes in turbidity measured by light transmission in the aggregometer (Aggr) and for O-RMS and light transmission with the rheo-optical analysis (RA) using the above DPDA method, are shown in FIG. 4, typically for human citrated (10 mM) platelet-rich plasma activated with 10 μM adenosine diphosphate.

With further reference to FIG. 5, an artery 50 has an artery wall 52. Flowing blood 54 travels along artery 50. A thrombus 56, in the FIG. 5, of roughly 1 cm in length forming an 80% occlusion of the artery 50, extends from artery wall 52. The shear rate, G that the blood in the occluded artery 50 is exposed to varies dramatically with respect to its location in the artery.

The flowing blood 54 approaching the thrombus 56 may typically have a shear rate of 2,000/s⁻¹. As the flowing blood 54 passes thrombus 56, the shear rate may rise to 10,000/s⁻¹ and immediately downstream of the thrombus may be as low as 50/s⁻¹. The time that the blood is exposed to the shear rates is also illustrated in FIG. 5. The blood is exposed to the very high shear rates for only short time periods in terms of msec. While the blood is then exposed to the shear rates for much longer periods, measured in terms of minutes.

In the assembly illustrated in FIGS. 1 and 2, the outer cylinder 14 and inner cylinder 16 may collectively form a cartridge releasably secured in housing 30. These cartridges being disposable after use. Different cartridges may exhibit a different width for annular chamber 18.

Different samples under investigation are introduced into the annular chambers 18 of the individual units 12 through the respective inlets 34, 36a, b via valve 37, or from sampling ports 24a, b via valve 27.

The inner cylinders 16 of each unit 12 are rotated by a motor 22 in a pulse fashion to mimic the variations in shear rate typically occurring in a pathological artery as described by reference to FIG. 5. Variations in the shear rates can be developed by employing cartridges in which the annular chambers 18 have different dimensions.

As the sample is subjected to the pulse rotation whereby the sample is subjected to shear rates which mimic the conditions of the fluid from which the sample is derived, in the body, the sample may be investigated such as by passing a light beam through the sample as illustrated in FIG. 3 and developing signals from which information is derived as to parameters of the sample such as formation of aggregates in the sample.

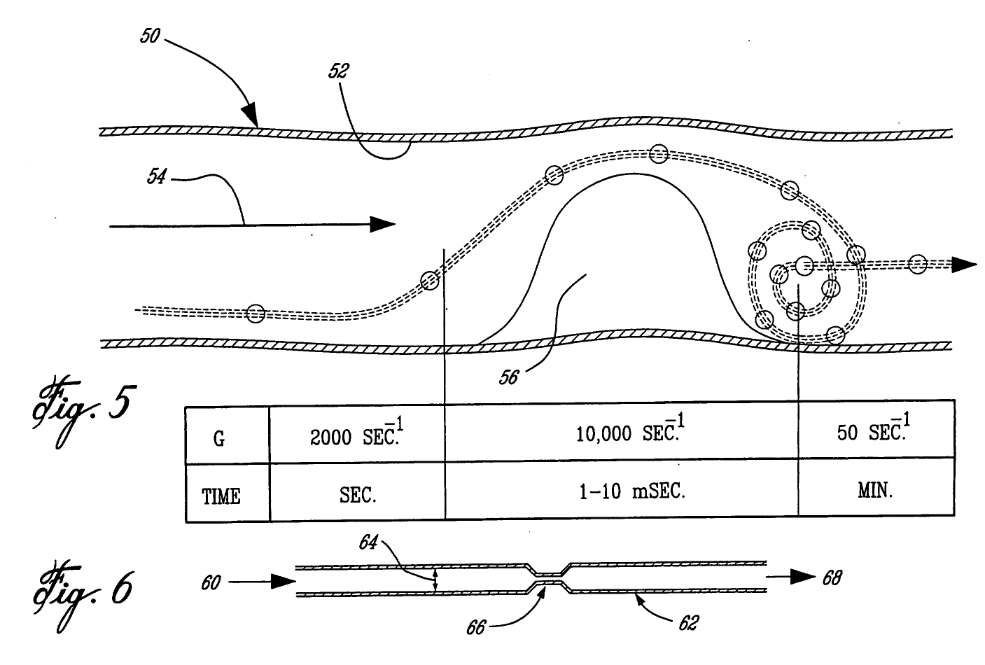

With further reference to FIG. 6 which illustrates a tube flow chamber which is a preferred embodiment of the present invention. The flow chamber comprises a tube 62 of diameter 64, with an inlet 60 and outlet 68 having therebetween a restriction 66. The flow chamber can be optionally attached at the inlet 34 or the outlet 25 of the microcouette. In a further embodiment the flow chamber may replace the microcouette entirely.

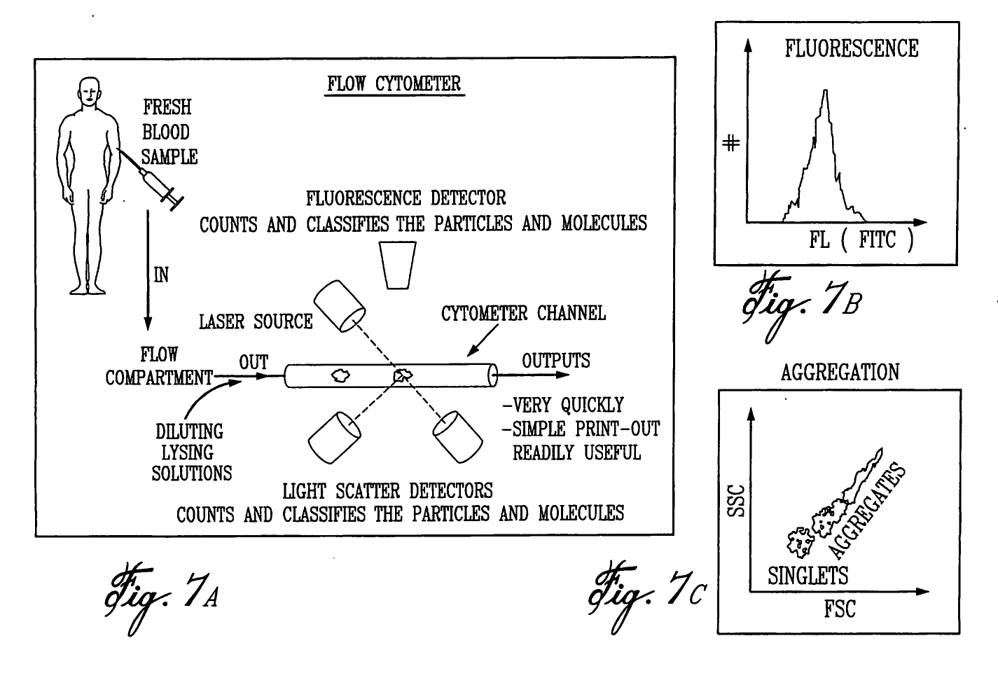

With further reference to FIG. 7A which illustrates the sampling process path, the method of detection and the results obtained according to the invention. The sample is obtained from a patient, it is injected into the flow compartment, diluting lysing solutions are added, within the flow cytometer the sample is subjected to a laser source with detectors that count and classify the particles. The outputs from the detectors are represented in FIG. 7B and FIG. 7C.

FIG. 7B represents the fluorescence signal generated by FITC (Fluorescein Isothiocyanate Chromophore) which is attached to a particle under observation and lit by the incident laser light beam versus the number of particles analyzed by the laser light hitting each particle observed in the flow cytometer.

FIG. 7C represents another output obtained of the FSC (Forward Scattering) versus SSC (Side Scattering) of light which is measured with detectors placed normal and perpendicular (respectively) to the incident laser beam lighting the observed particle in the flow cytometer.

By way of example, assembly 10 can thus be employed to investigate the efficacy of different drugs. Thus a different drug may be added to each sample, and the investigation carried out to determine how the different drugs affect formation of aggregates in the sample.

In this way a realistic indication can be obtained as to how the drug will behave in vivo.

### BACKGROUND PHYSIOLOGY AND FLOW CONSIDERATIONS

1. Range of Shear Rates in Normal and Pathological Settings:

The rate and extent of the aggregation in flowing suspensions in vitro and in the circulation in vivo are governed by the local flow conditions. Near-stasis conditions are found in separated flow in diseased vessels at bifurcations, known as “disturbed” flow, which is neither laminar nor turbulent. Time average values of wall shear stresses in the normal blood circulation found in post-capillary venules range from approximately 1 dyne/cm² to 20 dyne/cm², corresponding to shear rates of about 25-500 s⁻¹. Values of 500-5000 s⁻¹ have been reported for small arteries/arterioles, and mean values around 1700 s⁻¹. In “pathological” vessels, wall shear rates readily exceed 10,000 s⁻¹, generated at the top of plaques occluding the lumen of diseased coronary arteries by 50→80%.

2. Biological Effects of Shear:

Shear rates (G), and associated shear stress, can have multiple effects on aggregation by regulating i) the collision frequency between the cells in the suspension; ii) the lifetime of formed bonds (reverse rate constant),and (iii) the efficiency of cell adhesion which generally decreases with increasing shear rate for adhesive molecules whose structure and adhesive properties do not vary with shear stress. A significant alteration in the shear dependence of capture efficiency on shear rate is expected with shear sensitive receptors and ligands such as GPIb and vWF. The shear rate determines the duration of contact between two colliding cells, given by 2.6/G, with a critical time required for adhesive receptor/ligand organization and cross-bridging allowing ‘capture’ of two cells before they are forced to separate by the acting shear stresses. As the mean contact time between colliding particles decreases with increasing G, the efficiency for a given pair of receptor-ligands normally decreases with increasing G, unless shear dependent molecular conformational changes favoring new or enhanced molecular interactions occur. Thus the capture efficiency will reflect variations in number, types and conformations of ligands and receptors associated with the capture of cells or model latex spherical particles containing surface-immobilized adhesive molecules.

In the case of blood platelets, at pathological shear rates, typically >6000 s⁻¹, shear stresses can also cause i) the release of ADP from dense granules which has been shown to be associated with high shear stress-mediated platelet activation and subsequent aggregation, likely arising from (ii) the activation of the GPIb receptor and/or von Willebrand factor (vWF), where vWF is present in plasma, but can also be released from alpha-granules. VWF can bind to either GPIb or activated GPIIb-IIIa, also associated with platelet aggregation.

A device for simulating animal fluid parameters in vivo should, therefore, allow a control and proper definition of i) low and high shear rates (G) and stresses (tau) precisely, for example, normal arterial G at 2000 s⁻¹ but pathologic arterial G at 10,000 s⁻¹; and ii) exposure times (t-G) of cells to shear, recalling that physiological to pathological t values can range from milliseconds to minutes, hours or days.

3. Rationale for Controlled Flow Studies:

The flow conditions used in cell/particle aggregation are important because shear conditions determine whether or not any aggregation will occur between otherwise potentially competent receptors and ligands capable of supporting adhesion. The size of the cell or particles is also relevant since a given flow creates more force on a large cell/particle than smaller cell/particle. Thus, a platelet aggregometer, which measures formation of large aggregates of platelets in stirred suspensions under ill-defined flow conditions (variable and ultra-low shear rates of about 10-75/s⁻¹), although widely used in hematology labs for assessing gross changes in platelet aggregability does not give a reliable indicator.

4. Rationale for Using Cell Suspensions in Couette Flow Devices:

It may appear that collisions of particles in suspension cannot represent the collision of a particle with a immobilized planar surface because in suspension the two rotating particles undergo compressive and then tensile separating forces during one rotation. In reality, it can be seen that similar if not more complex forces operate during collisions of particles with a vessel wall. Thus, red cells can provide compressive forces as they force platelets to the surface from flowing blood; vessel walls and endothelial cells are not smoothly flat and indeed undulate with time, while leukocytes and activated platelets have respectively dynamically-changing membrane folds and pseudopodal projections, all of which may readily be associated with compressive and tensile forces occurring with cells colliding in free suspension.

In accordance with the invention cell adhesion to planar surfaces may be modeled by particle aggregation in flowing suspensions. Aggregation studies allow precise control of the interaction events per unit time. In the cases where studies are done with whole blood where red cells are necessary for driving particles like platelets to the surface under study for adhesion, special attention may be paid to possible activation artifacts of platelets and other cells by ADP released from the flowing and sheared red cells.

The operative shear stresses will depend on the local viscosity and in particular the local concentrations of red blood cells which should be characterized.

In the present invention a variable of shear stress at a given shear rate may be achieved by controlling a variable viscosity of the cellular environment, using “neutral” polymer solutions.

5. Rationale for Individual Particle and Cell Analyses:

Flow studies of cell adhesion desirably include the study of the behaviour of individual cells for a distribution of cells. The behaviour of individual cells may be examined by particle counting techniques even when assessing inter-particle aggregation. It is known that subpopulation responses of cells can exist to given concentrations of agonists or drug inhibitors. In addition, it is useful to determine the “molecular history and evolution” of adherent cells, and relate the number density and organization of relevant receptors and ligands to cell adhesive and migratory functions. Flow cytometry is appropriate to examine individual cells, including cells resuspended into single cell preparations from immobilized surfaces. Finally, suspensions as complex as whole blood can readily be evaluated for number concentration and size distribution of specific cells such as platelets without separating the cells.

6. Rationale for Evaluating Cell Adhesivity and Drug Inhibition at Different Shear Rates:

Flow devices need to reflect a physiologically relevant measurement of a hemostatic parameter, for example, bleeding times in a known type of vascular bed; and tests in flow devices need to be made in a wide range of shear rates, as drug efficacy will vary according to the shear rate, and associated molecular determinants centered on distinct ligands (Fg vs vWF) and receptors (GPIIb-IIIa and GPIb).

7. Rationale for Varied Pulse Flow Regime (VPFR):

Although all blood cells, including platelets, leukocytes, and metastatic cancer cells, are exposed to an enormous range of shear rates during normal physiology, from low venal to high arterial, presumably without pathological consequences, these cells can experience very rapid and transient jumps in shear rates (G) and associated shear stresses (Tau) in pathological vascular settings.

For example, as depicted schematically in FIG. 5, it has been estimated that wall shear rates may reach upwards of 10,000 s⁻¹ in highly stenosed arteries, with a number of sources reporting that platelet activation and spontaneous aggregation can be induced by shear rates in whole blood and in plasma respectively around 1000 s⁻¹ and 6,000 s⁻¹; these studies have normally been conducted with exposure times of 30 seconds to many minutes or hours, whereas calculations for a real arterial stenosis indicate transient times of the order 1-50 msecs within such high flows in stenosed vessels. In fact, it has been shown that a cell will experience shear rates of the order of 2,000 s⁻¹ in an artery prior to entering the stenosed vessel, then 10,000 s⁻¹ for 1-50 milliseconds, and subsequently very low shear rates for “long” times, i.e. the order of 50 s⁻¹ for many seconds to minutes due to flow separation post-stenosis (and indeed found in Y-junctions where atherosclerotic plaques develop over months to years of a human's lifetime); thus, cells may be “activated” and/or “primed” (made hypersensitive to subsequent chemical activation) by the millisecond exposure to “ultra-high” G values, and then further activated by the combined long residence times favoring local chemical accumulation and ultra-low G favoring highly efficient cell capture into aggregates and interactions with endothelial cells.

In particular embodiments it is proposed, in the method of the invention to pulse-shear cells at 6,000-10,000/s⁻¹ for 1-50 msecs, followed by shear-dependent studies of further chemical and or physical activation and/or aggregation at ultra-low G of 10-100/s⁻¹, and this may be readily pre-programmable by a computer-interfaced electronic control box in the apparatus of the invention.

The apparatus of the invention provides a novel combination which converts the known concentric coaxial couette device into a parallel set of multiple wells each with inner cylinders, but of varied diameters with electronically controlled pulsed and patho-physiologically-relevant varied flow regimes, shear stresses. The invention also provides novel methods of using control and reactive beads, new analytical methods, and computer software for simple data and parameter presentation.

8. Specific Embodiment:

In the invention natural cell to cell contacts for a variety of cells suspended in physiologically-relevant media are exploited. The invention is applicable to human disease problems as varied as cardiovascular, using blood cells like platelets, leucocytes and red cells; vascular biology, using endothelial cells; new technology whereby endothelial cells from clinical tissue sources made to grow on latex spheres are sheared in the apparatus of the invention with other blood cells or cancer cells; metastatic diseases using different metastatic cell lines presented in suspension to each other and to endothelial cells and selected blood cells; inflammation and sepsis, using different bacterial strains; blood-borne diseases such as parasitized red blood cells; and many more situations where flowing cells have adhesive capacities that need to be characterized.

In addition, platelet and blood cell function need to be evaluated in ex vivo flow systems for clinical procedures including deleterious effects of bypass surgery, of surgical procedures, and blood banking operations such as platelet storage, as well as for the flow-dependent evaluations of the use of particle vectors like liposomes, latex spheres or viruses/plasmids in drug and gene delivery therapies. The invention has potential applications in colloid systems related to industrial processes and products as diverse as in the pulp and paper, food and cosmetic industries, and in evaluating the particle properties in suspensions of complex heterogeneous mixtures of polymers, particles or cells.

By means of the invention it will be possible to evaluate not only patho-physiologically altered adhesive states of cells, both native and invasive to the human organism, but to evaluate potentially bedside efficacy of anti-adhesive drugs, especially where dosage must be critically evaluated due to large and dangerous interdonor variations (e.g. oral antithrombotic drugs targeted to the GPIIb-IIIa platelet adhesive receptor).

A simple parameter of cell adhesivity independent of cell concentration, the adhesion efficiency, as well as size distribution of aggregates, but as a function of shear rate, stress and exposure time.

The invention integrates into a flow device system, for the first time ever, a time-varied regime of shear stress (controlled by shear rate angular velocity and geometry of inner cylinder) and local viscosity, which can be controlled with select concentrations of “inert” polymer solutions, as well as computer-generated pulsed shear rate variations mimicking pathological settings. Thus the apparatus may employ, for example, a 1-10 msec pulse of shear at 5,000 to 10,000 s^{31 1}, followed by a longer pulse of arterial shear rate at 10,000 s^{31 1}, as well as the known very low shear rates existing in separated flow in arterial bifurcations (down to a few per sec shear rates, favoring highly efficient cell adhesion and aggregation).

Existing experimental approaches using many minutes of exposure times of platelets to ultra-high shear rates (10,000 s⁻¹) are not relevant to abnormal human cardiovascular patho-physiology.

In the invention, it will be possible to determine the capture efficiency dependence on shear rate, which reflects cell adhesive participating surface molecules pathological or drug disturbances, and this by selecting a variety of stable shear rates varied in different microwells of the apparatus with select inner cylinders, running simultaneously in parallel samples, and varied over the range of venal to arterial flow conditions depending on the “disease” state, being evaluated.

Computer processing and print-out of particle adhesive efficiency, aggregate size distribution, and time-dependent reversibility can be determined, with the invention, as a differential function of a time-dependent variation in shear rate, with a super-imposed pulsed ultrahigh but short-life shear rate, e.g. 10,000 s⁻¹.

A 37° C. temperature control of the device will permit many advantages over doing room temperature studies.

Subpopulation behaviour is relevant to cell heterogeneity as well as heterogeneous responses of cells to drugs. In different embodiments of the invention particle counting and single cell flow cytometric analysis miniaturized by dynamic particle counting may be incorporated in the apparatus, by pulsing cells into a flow cytometer, for example, an ultramicro and dedicated flow cytometer built into the apparatus for each unit.

### DETAILS FOR MULTI-TECHNIQUE INTEGRATION INTO APPARATUS

A) A microcouette flow device allows the shearing of micro-samples of suspensions of particles and cells at homogeneous shear rates and shear stresses, but with electronically-controlled and pre-programmed variations in time and in flow regimes simulating pathological settings for abnormal cell adhesion in disease. Both chemical and shear-stress-induced activation of cells can be studied in the device.

Shear stress can directly activate, cells via shear stress receptors as present on endothelial cells, or via physically altering extracellular membrane proteins to induce conformational changes leading to altered shear-induced cell adhesivity. Shear rates and stresses can be pulsed and varied in time and magnitude by pre-programmed control of angular velocities developed by the motor, as well as by varying the viscosity of the suspending medium with “inert” high molecular weight polymers (specifically, varying viscosity from 1-5 centipoise, recognizing that whole blood viscosity can vary from about 1-2 cp in plasma free of red cells near some vessel wall surfaces, to 4-5 cp when rich in flowing red blood cells).

Addition of select agonists according to the disease state being evaluated (e.g. ADP or thrombin in platelets physiology, or cytokines in inflammatory responses or metastatic cell behaviour). Chemical agonists can either be pre-added to the suspensions prior to shear, or can be directly injected into the sample during shear.

B) Rheo-Optical Systems at Three Levels:

Fiber-optics and detectors for measuring light passing through the suspension within the well between the two concentric cylinders, for measuring real-time dynamic changes in particle number and size distributions, with the DPDA as preferred option using detector 28 and light guide 26, and secondary choices of optics for two options; detector 128 for turbidity measurements for transmitted light (TA) using forward scatter (FSC), and light guide 26a for dynamic focal point light scattering (DLS).

C) Particle Concentrations:

Cell suspensions can be studied at three regimes of particle concentrations, depending on the parameters being evaluated: a) high concentrations, typically at particle counts around 400,000/μL, allowing rapid large aggregate formation, and detected by simple bulk light transmission (TA; macroaggregation); b) intermediate concentrations, typically at particle counts around 40,000/μL, allowing kinetic measures of microaggregation (PA) and associated adhesion efficiencies; and c) low concentrations dilute enough to minimize particle aggregation during shear, allowing rheo-activation studies (shear-induced activation of cells in absence of aggregation). However, undiluted whole blood studies can be performed to measure typically both PA and TA, as well as single particle measures of, for example, blood platelet aggregate number and size distribution using flow cytometry (FIG. 7).

D) Microflow cytometry with standard 480 nm excitation and emission from fluorescently-labeled markers can allow measures of single and aggregated particle size distributions, as well as surface composition. A built in miniaturized and dedicated microflow cytometer per microwell under shear; as well as an option for pulse ejection of microsamples fed directly on line to large commercial flow cytometers.

E) Microcarrier Bead (MCB) Presentation of Adhesive Molecules or Cells.

F) Microassays on Single Cells (MASC).

G) Magnetic bead technology (MBT) for concentrating and capturing magnetic beads made to interact with cells or present as magnetized MCB in the above studies.

### EXAMPLE

A typical multiple-well, multiple-gap, pulsed shear rheo-optical apparatus of the invention is illustrated in FIGS. 1 to 3, for measuring cell activation and adhesiveness in microsamples with programmable pulsed flow in real time.

Cell or particle suspensions flow into plastic (and for other materials such as stainless steel) microtiter wells (chamber 18) of up to 25 wells in an assembly 10, each well having an outer diameter of about 11 mm (outer radius Ro=5.5 mm), with a first model containing 4 wells per assembly 10, then scaled up to a second model with a 5×5 array of similar sized wells (fixed Ro). Inner concentric cylinders 16 with diameters of 10 to 10.4 mm (Ri=5.0 to 5.2 mm) are coupled (platformed) to a central drive (motor 22) for one motor control, or to individual motors (dedicated), for driving and pulsing a time-dependent range of shear rates and stresses, simulating the range of patho-physiological variations in human cardiovascular disease. Thus the gap (width of chamber 18) will be selectable between 0.3-0.5 mm, generating a five-fold range of shear rates at any given angular velocity (w) of the inner cylinder. Thus, different wells or chamber 18 can be set up to generate distinct shear rates and associated shear stresses by selecting different inner coaxial cylinders 16, all at the same angular velocity. The bottom of the inner cylinder 16 has a conical end 32 with an angle theta chosen so that cells below the cone will experience the same uniform shear rate as in bulk suspension between the coaxial cylinders 14 and 16. A brief, electronically-controlled initial pulse of 1-50 msec at ultrahigh shear (typically 7500/s⁻¹) “pre-conditions” cells in a manner predicted to occur pathophysiologically, whereafter shear studies are carried out on cell function at either normal arterial (about 1000-2000/s⁻¹) or ultra-low shear found in separated flow at atherosclerosed or stenosed vessels (about 50/s). The medium viscosity can be readily changed from 1-5 cp with Ficoll polymer solution to yield “equivalent” shear rates up to 5-times higher at any given velocity:

Rheo-Optical Systems at Three Levels:

Level I: Fiber-optics and detectors for measuring light passing through the suspension within the well between the two concentric cylinders, for measuring real-time dynamic changes in particle number and size distributions, with the DPDA as preferred option, and secondary choices of optics for two options; detector 128 for turbidity measurements for transmitted light (TA) using forward scatter (FSC), and light guide 26a for dynamic focal point light scattering (DLS) (U.S. Pat. No. 5,907,399, Shirasawa Y. et al., May 25, 1999).

Dynamic focal point light scattering (DLS) allows real-time measurement of particle aggregation rates and extent, as well as mean sizes for formed aggregates, possible with dilute suspensions, made possible by selecting a micro-area for analysis, a region of about 33×65x×65 micrometers and a laser light column about 33 μm wide. It has been demonstrated that as little as 30 human platelets in blood plasma can be detected in a measured microvolume of 0.00014 cc, with a sensitivity between 0.5 to 10 μm sized-particles. Light transmitted through a column of suspensions, on the other hand, may be monitored to follow cell macroaggregation as currently used in classical aggregometry, but in the present invention the measure would be rheo-aggregometry at well-defined flow rates.

Level II: microflow cytometers built into each outlet, or alternating rapidly between outlets, in plug flow cytometry, whereby micro-subsamples are delivered as small boluses or plugs of cells for flow cytometric analysis. For more sophisticated studies, fluorescence markers and direct interface with a large commercial flow cytometers, such as the Becton-Dickinson models, routinely found in large hospitals, can be used. This modification allows data accumulation for sub-population properties, with output for individual cells/aggregates or particles, and also permits direct measures within sheared cell mixtures as complex as whole blood.

Aggregate stability may also be tested, where disaggregation can be followed as a function of time and shear stresses, by imposing a programmed regime of increasing shear stresses by varying the gap (width of chamber 18), the angular velocity and or the medium viscosity.

In a further embodiment the assembly 10 may have a larger number of units 12 allowing up to a hundred samples per assembly to be sheared, especially in rheo-activation studies where very dilute suspensions down to single cells can be evaluated.

A rectangular or concentrically-arranged circular array may be employed in assembly 10 for studies of cell activation and aggregation down to a few cells per individual units of a microchip array

9. Target Users:

The assembly 10 has both diagnostic and pharmacodynamic applications with the latter being of particular importance.

A) Thus, altered responsiveness of cells to shear and chemical activation in disease states may allow diagnosis of high risk patients for cardio-cererebro-vascular and for metastatic diseases; B) Altered responsiveness of cells to shear and chemical activation following drug intervention in a variety of cardio-cerebro-vascular diseases, and metastatic cancer cell diseases, can be assessed in patho-physiologically-relevant flow conditions.

Disease States and Associated Cells which may be Evaluated Include:

Circulating blood cells, including normal and pathologic red cells (e.g. malaria infected rbc): leukocytes, especially during inflammation; platelets associated centrally with hemostasis and thrombosis, and occlusive thrombo-embolic problems in cardio- and cerebro-vascular ischemia; and organ dysfunction (heart attacks and strokes, as well as sequalea of cerebral occlusion and life-threatening subsequent hemorrhage).

Tumours which enter the blood circulation, mimicking adhesive interactions seen with leukocytes/platelets and endothelial cells, as strategy for targeting particular vascular sites and extravasating to establish new secondary tumours and associated angiogenic activities.

Bacterial adhesion studies underlying septicemia.

Inflammation and leukocyte/platelet/endothelial cells.

Respiratory—epithelial cilia sweeping motions in lung diseases.

Sperm motion in flow.

Evaluation of antithrombotic drugs ex vivo is important in attempts at developing oral agents, where interpatient pharmacodynamics puts patients at high risk for deathly bleeding, and where the drug companies and clinicians recognize a desperate need for bedside devices for monitoring the efficacy of the drug administration.

### SUMMARY OF PREFERRED EMBODIMENTS

1.1 Overall Description

The device of the invention is a microflow device allowing dynamic, real-time measurements of adhesion and aggregation of microsamples of suspended polymers, particles or cells as a function of patho-physiologically relevant flow variations. In the case of cells underlying human diseases, such as platelets and leukocytes in cardiovascular disease, and both blood and metastatic cancer cells in oncology, pathologically-relevant flow conditions can be evaluated for both prognostic and follow-up after-drug treatments.

The device will integrate light measurements with precisely-controlled flow conditions, for determining bulk properties of aggregating cells or particles, additional embodiments will incorporate modular features which allow sophisticated measures of fluorescent probes associated with specific cells and biological molecules, and also subpopulation behaviour of the aggregating cells, more detailed analyses of particle size distributions, and possibilities of studying mixtures of cells or particles in suspension as complex as whole blood.

A) The new flow device, integrates the dynamic measurement of aggregation rates, extent and overall aggregate size, with a patho-physiologically relevant range of precisely controlled flow rates at physiologic temperature (37° C.) by using:
a) dynamic photon dispersion analysis (DPDA) (FIG. 3): real-time measurement of particle aggregation rates and extent, discernable for early onset micro aggregation (doublets to microaggregates containing only 3-10 platelets per aggregate), as well as for more advanced macroaggregation (>20 platelets/aggregate).
b) geometrically and mechanically-imposed flow changes, a disposable flow cartridge containing the inner and outer concentric cylinders can be i) selected for different gap dimensions determining the operative shear rate at any given angular velocity of rotation of the inner cylinder, and ii) the angular rotational velocity will be imposed with an electric motor whose speed and duration will be computer-controlled, with a functional range of speeds and time varying from about 10 to 10,000 rpm, and msecs to many minutes, respectively.
c) dynamically-injectable particle suspensions; or chemical activators or inhibitors, can be introduced directly into the flowing suspension;
d) housing of the disposable flow cartridge at 37° C. for physiologic temperature and regulation of suspension viscosity (which changes shear stresses at a given shear rate);
e) computer-controlled programming of flow and microinjection conditions, for a variety of pathologically-relevant study conditions, e.g., shear-induced particle alteration/aggregation (SIPA) at ultra-high flow rates (7000-10,000 per sec) without need for extrinsically-added activators; or activator and shear-induced platelet aggregation (ASIPA) determined at a selected combination of time-dependent flow variations;
f) print-out of key adhesive and cell aggregation parameters through on-line computer interfaced computations: initial rate (=capture efficiency) and time-dependent extent of microaggregation, as well as rate and time-dependent extent of macroaggregation.
B) The device may also allow real-time measurements of chromophore emission from chromophore-labelled polymers, particles or cells, and in this embodiment will contain an intense light source at distinct wavelengths (typically He—Ne laser at 480 nm) for exciting relevant chromophores within the flow device, and measuring transmitted light intensity at the relevant emission wavelength.

This device allows the studies of reporting chromogenic substrates for measuring for example protease activities or fluorescently-labelled reporters for a wide variety of cell functions, in cell suspensions varying from isolated platelets or cancer cells to cell mixtures as complex as whole blood.

C) A further embodiment allows individual particle analysis and will contain:
a) microflow cytometry (MFC): for single and aggregated particle size distributions, as well as surface composition. Built-in miniaturized MFC per microwell under shear or pulse ejection of samples to a free standing flow cytometers;
b) particle counting: particle concentrations in three regimes for measure of i) rapid large aggregate formation (TA), ii) micro aggregation (PA) and iii) rheo-activation studies (shear-induced activation of cells in absence of aggregation).

The flow devices of the invention, are based on the couette device which evaluates viscosities of water and oils

1.2.1 The Flow Compartment:

The device in a simple embodiment employs duel-wells, fixed-gap, pulsed shear rheo-optics for measuring cell activation and adhesiveness in microsamples with programmable-pulsed flow in real time. The multiple flow chambers will allow simultaneous parallel studies of a plurality of samples, with the important possibility of studying “controlled” and “perturbed” samples e.g., activated platelet suspensions with and without added drugs. Or compared at distinct shear regimes.

Cell or particle suspensions are made to flow into the space between two coaxial cylinders (14, 16) constituting the flow chamber 18, with an outer diameter of typically 11 mm (outer radius Ro=5.5 mm). The inner concentric cylinder 16 with diameter typically of 10 mm (Ri=5.0 mm; and a resulting gap of 0.5 mm) is coupled (platformed) to a central drive motor 22 for one motor control for driving and pulsing a time-dependent range of shear rates and stresses, simulating the range of pathophysiological variations in human cardiovascular disease. Rotation of the inner cylinder 16 generates a fixed shear rate at any given angular velocity (w), with a range of 10-10,000 s⁻¹ readily achievable with a very small commercial motor (4.4×2.4 cm sized servomotor). The cell suspension is pumped into the cylinder space 18 via an inlet 34 at the bottom of the outer cylinder 14.

The cylinder height of 10-30 mm will allow use of only 100-400 microliters of cell/particle suspensions. Typically, one ml of anticoagulated blood is added to a well 31 in FIG. 2 in the device, and a small volume is either used directly handling whole blood analyses, or the platelet-rich plasma (PRP) which either spontaneously forms with sedimentation times of only a few minutes (or via built-in well controlled microcentrifugation), is pumped out via a catheter into the flow cartridge 18. A catheter or needle 40 feeds inhibitors/activators/further particles or cells (1-10% by volume) into the flowing suspension.

The bottom 32 of the inner cylinder 16 has a conical shape, preferably with an angle chosen so that cells below the cone will experience the same uniform shear rate as in bulk suspension between the coaxial cylinders (14, 16).

In a preferred embodiment a tube flow chamber 62 illustrated in FIG. 6, can be coupled to or may replace the microcouette. The tube flow chamber 62 when coupled with the microcouette can be placed directly after the pump 38 or at the outlet 25 after valve 27. The tube flow chamber 62 comprises a tube of inner diameter 64 with a diameter varying between 0.3 and 2.0 mm and similar to the width of the annular space 18 of the microcouette. The tube 62 may be homogenous or contain a short restriction 66 of diameter typically 0.1 mm, this when coupled to a variable speed pump would simulate the range of shear rates and shear stresses as previously described for the case of the microcouette. The ratio of the inner diameter 64 to the diameter of the restriction 66 is between 2 and 10 and preferably 5. All other aspects including the analytical measures of cell aggregation or fluorescent markers, including the coupling to microflow cytometer (FIG. 7A) are the same as described for the microcouette embodiment. Furthermore the tube flow chamber can be placed in a variety of configuration such as a flow through one pass mode or be arranged in a flow loop or coil.

1.2.2 The Flow compartment: a Disposable Shear Cartridge

The practical utility and commercial interests of the device are greatly enhanced by the disposable feature of the entire flow chamber which will be housed within a temperature-controlled (37° C.) metallic unit, with optical probes and detectors built permanently into the supporting case for the flow chamber. The “disposable” flow chamber will contain the inner and outer cylinders, readily decoupled from the driving motor and the metal casing, henceforth known as the “shear cartridge”. This disposable unit is expected to also contain the chemical solutions (ADP and Thrombin mimetic) used for injection and activation of the sheared samples. Thus the “shear cartridge” disposability will ensure reproducibility of results for distinct samples.

This readily disposable feature will also readily allow the introduction of flow chambers containing gap widths of 0.3 to 1 mm, depending on user needs, with the range of corresponding shear rates for any given rotational angular velocity of the inner cylinder varying over a 3.3-fold range.

The basic four cartridge device may be readily expanded to contain from upwards of 4-10 shear cartridges per instrument. This would permit parallel studies for example of the effects of a number of different drugs on blood samples of any given patient, useful in appropriate bedside or pre-treatment drug selection for any given patient. Further miniaturization of the shear cartridges is optional, though readily accomplished.

The tube flow chamber 62 embodiment of the invention can also be made to be disposable.

1.2.3 Computer-Programmed Flow Modes

The device will typically have at least two preprogrammed “clinical” modes, called the thrombosis and stenosis modes; on-site custom programming, as well as a aggregate stability mode.

In the thrombosis mode, a brief, electronically-controlled initial pulse of 1-50 ms at ultrahigh shear (typically 7500/s⁻¹) will “pre-condition” cells in a manner predicted to occur pathophysiologically, followed by shear studies of cell function at either normal arterial (about 1000-2000/s⁻¹) or ultra-low shear found in separated flow at atherosclerosed or stenosed vessels (about 50/s⁻¹). Thus, ultra-high shear preconditioning is followed by chemically-induced, shear-associated studies of platelet aggregation (CSIPA).

In the stenosed mode, there will be a programs to assess pure shear-induced platelet aggregation (SIPA), typically evaluated at 7,000-10,000 s⁻¹ in plasma free of red blood cells, but at about 2000 s⁻¹ in whole blood; where no extrinsic chemical activators need be added, and free select features for programming both duration and shear rates are to be imposed.

The “on-site custom programming” mode will allow easy settings for a choice of flow regimes, duration of applied flows, and choices of activator/inhibitor concentrations. One likely recommended mode will be to assess platelet aggregation dynamics at 300 versus 2500 s⁻¹ shear rates for two parallel samples to obtain a fingerprint behaviour of the shear dependence of behaviour of a platelet preparation being evaluated for example for effects of a GPIIb-IIIa receptor antagonist.

A shear-induced threshold determination mode, provides a programmed step-wise increase in shear rates from about 100 to 7500/s⁻¹, for measure of the threshold of shear for onset of aggregation, occurring in the absence (uniquely shear-induced) and presence (shear plus chemical activator-induced) of low chemical activator conditions.

A further embodiment will permit testing of aggregate stability, where disaggregation can be followed as a function of time and shear stresses. The above approach will be simply modified to impose a programmed regime of increasing shear stresses by varying the angular velocity of the rotatable inner cylinder at any given medium viscosity.

In addition, the medium viscosity may be changed from 1-5 cp with Ficoll polymer solution to yield a) viscosities closer to that of flowing blood, while b) enhancing the operative shear stresses at any given shear rate (as stress=rate×viscosity).

A) Simplest modes: i) pre-mix mode: Cell suspensions are premixed in a microstir chamber with activator or inhibitor (1-10% by volume) and pumped slowly into the shear cartridge prior to onset of shear; or, ii) A simpler solution consisting of 1-3 entry ports built into the external cylinder and direct infusion into these ports, especially in the area of optical observation near the center of the device.

B) Direct Infusion into Flowing Suspensions within the Shear Cartridge:

While a given sample is being sheared, and following optical probing of a baseline, an activator and/or inhibitor will be instantaneously introduced into the flowing suspension by microinjection technique: thus, effects of activators on inducing shear-dependent cell activation and aggregation, as well as effects of inhibitors added before, during or after aggregation, can readily be tested in real-time on the flowing cell suspensions.

A catheter with multiple openings or a long solid metal needle (<0.4 mm outer diameter) is connected to the chemical activator/inhibitor solution via tubing and a micropump. The catheter or needle is programmed to rapidly move towards the bottom of the flow chamber (either vertically or at a sharp angle through an insertion near the top of the outer flow cylinder), and following its maximal insertion, the solution will be pumped into the chamber already containing the flowing cell suspension, at a constant flow, but in the case of the needle, with its simultaneous rapid withdrawal, to generate about 1-10% by volume instant mixing (induced by the rotating fluid). If mechanical distortion problems arise due to the operative shear, the flow can be interrupted during the injection period, with mixing occurring instantly with flow resumption.

1.3 The Rheo-Optical Systems

The Device will Typically have Rheo-Optical Systems at Three Levels:

I) A highly informative detection mode, using a well-established optical technique called dynamic photon dispersion analysis (DPDA) (J. Gregory, Turbidity Fluctuations in Flowing suspensions, J. coll Interf Sci 105:357-71, 1985). Significantly, this technique has been used widely in colloidal and non-biological particle flocculation studies, but not in biomedical sciences to-date.

II) A second modular option allowing excitation of chromogenic probes present or expressed on/in particles or cells or in solution, monitored from light emission, but as a function of flow conditions; and

III) A third option that can be added to system I, requiring individual particle histograms in terms of particle concentration and particle surface molecular properties, this employs a flow cytometer.

1.3.1 Level I: Optical System

Dynamic photon dispersion analysis (DPDA) is used to measure the light transmitted through a horizontal cross-section for a narrow light beam (0.3-0.4 mm) passing proximal to the stationary outer cylinder of the microcouette device, without touching or penetrating the rotating inner cylinder, with an estimated light path of about 1-3 mm (FIG. 3). A simple LED of about 1 mW and 820 nm is used via 26 with a sensitive PIN photodiode 28. The metal housing containing the flow chamber also contains the LED and p hotodiode detector, with a 0.4 mm drilled hole allowing passage of the 0.4 nm -wide light beam through a cross-section between the inner and outer cylinders, entering at mid-height of the sheared suspension. For very complex suspensions, such as whole blood, the LED can be replaced by a more intense light source, such as a He—Ne laser typically at 480 nm.

The LED is driven from a constant-current source (50 mA), and the signal from the photodiode is passed to a high-impedance amplifier, whose output is monitored to give the mean transmitted light intensity i) below) (typical reading of 10 V for water). The output is also ac-coupled (via a capacitor) to a further amplification stag so that the fluctuating component of this output (a few mV) can be isolated and amplified. This fluctuating signal is passed to an rms-to-dc converter, whose output is a voltage equal to the true rms value of the input signal.

The transmitted light intensity, measured as a voltage output from a photodiode detector, is measured for:
i) changes in fixed DC voltage level, corresponding to the turbidity of the suspension in shear (Δτ in tracing RA, rheo-optical analysis) in FIG. 4, and related dynamically to large platelet aggregate formation (>20 platelets per aggregate), as currently optically routinely measured in the classical platelet aggregometer for measuring macroaggregate formation (Aggr. tracing in FIG. 4); and
ii) changes in the oscillations about the DC voltage, expressed as root mean square values (O-RMS) (as in FIG. 4). Reflecting both particle number and size of the moving particles in flow. These O-RMS values are exquisitely sensitive to onset of microaggregation (single platelets forming doublets and early-order aggregates (<10 platelets per aggregate), allowing sensitive dynamic measures of initial rates of microaggregation, as well as extent and stability of microaggregate formation. Note that the current classical aggregometer totally misses microaggregation with current optical systems and geometries used, while also being limited to one very low and ill-defined flow regime.

1.3.2 Level II: Optical Module for the Excitation/Emission (XM)

In addition to the simple DPDA optical system in Level I the device may include an option consisting of an intense light source at distinct wavelengths (typically He—Ne laser at 480 nm) for exciting relevant chromophores within the flow device, with excited emitted light measured with appropriate filters and photomultiplier tubes (PMT). The exciting and emitted light beams are positioned on the opposite side from the DPDA and about one-third elevation from the bottom of the flow device, but similarly housed permanently within the metal housing containing the DPDA and “shear cartridges”. This device allows the studies of reporting chromogenic substrates for measuring for example protease activities or fluorescently-labelled reporters for a wide variety of cell functions, in cell suspensions varying from isolated platelets or cancer cells to cell mixtures as complex as whole blood.

1.3.2 Level III

A microflow cytometer is built into an outlet of the device, for samples withdrawn via a micropump through a tube external to the flow device (FIG. 7), or alternatively a coupling of the withdrawn sample will be made to a free-standing flow cytometer, whereby micro-subsamples are delivered as small boluses or plugs of cells for flow cytometric analysis. For more sophisticated studies, fluorescence markers and direct interface with a large commercial flow cytometers, such as the Becton-Dickinson models, routinely found in large hospitals, may be used. This modification allows data accumulation for sub-population properties, with output for individual cells/aggregates or particles evaluated within cell suspensions as complex as whole blood.

1. a method of determining a parameter of polymers, particles or cells in a liquid vehicle comprising:
a) introducing a sample of a liquid vehicle containing suspended polymers, particles or cells into a chamber comprising an outer cylindrical wall including an arcuate inner surface;
b) subjecting said sample, to flow and shear conditions within the chamber and along the inner surface of the outer cylindrical wall to up to 10,000 sec⁻¹, and
c) exposing said sample, under said flow and shear conditions in said chamber, to at least one polymer, particle or cell parameter determining operation,
wherein said suspended polymers, particles or cells are analyzed by flow cytometry for numbers of agglomerates per unit volume, as a measure of particle aggregation, the sample being analyzed from forward scatter (FSC) and side scatter (SSC) of each particle flowing past a reporting laser light beam.

2. A method according to claim 1, for determining a parameter of cells in an animal body fluid wherein said sample in a) is of an animal body fluid containing suspended cells,
wherein said sample is subjected to said conditions in step b) in the presence of an activator or inhibitor for said cells; and step c) comprises transmitting a light beam from a source through said sample, to a detector, developing a signal responsive to the detected light at said detector and evaluating a cell parameter from said signal,
wherein said signal is in the form of a voltage output and changes in voltage are detected, as a measure of turbidity of the cells under said conditions, said turbidity being a measure of large aggregate formations thereby providing a measure of macroaggregate formation in the cells under said conditions in the presence of said activator or inhibitor.

3. (canceled)

4. (canceled)

5. (canceled)

6. A method according to claim 1, wherein chromogenic substrates for measuring protease activity, or fluorescently-labelled reporters for cell functions are detected by transmitting a light beam from a source through the sample to a receiver for the excited light, and the received light is monitored as an indicator of molecular changes at cell surfaces or in solution association with suspending and flowing cells, such as the production of proteases, for example, thrombin.

7. A method according to claim 1, further including withdrawing at least a portion of the sample from said chamber subsequent to step c) and subjecting the withdrawn sample to flow cytometry.

8. A method according to claim 1, wherein said chamber is an annular chamber defined between an outer stationary cylinder and a rotatable inner cylinder and said conditions in b) are produced by rotating said inner cylinder relative to said outer cylinder, under a pulsed rotation condition.

9. A method according to claim 1, wherein step a) comprises introducing a plurality of samples of the animal body fluid, each into a separate annular chamber of a corresponding plurality of annular chambers, each separate annular chamber having a width being defined between a separate stationary outer cylinder and rotatable inner cylinder; and the rotatable cylinders are driven collectively by a single common motor,
wherein the width of each separate annular chamber may vary.

10. (canceled)

11. (canceled)

12. A method according to claim 2, including fluorescently labelling the cells in said sample, prior to introduction of the sample into said chamber, and analyzing cell types and molecular markers on cell surfaces.

13. A method according to claim 1, wherein said chamber is placed in series with a tube flow chamber.

14. A method according to claim 1, wherein said chamber is a tube flow chamber fed by a variable speed pump.

15. A method according to claim 13, wherein the tube flow chamber has a diameter between 0.3 to 2.0 mm.

16. A method according to claim 13, wherein the tube flow chamber includes a restriction of diameter 0.05 to 0.2 mm diameter.

17. A method according to claims 13, where the tube flow chamber is selected from the group consisting of a single pass, a flow loop and a flow coil.

18. An apparatus for determining a parameter of polymers, particles or cells in a liquid vehicle comprising:
a) a housing defining a chamber having an outer cylindrical wall including an arcuate inner surface for receiving a sample of the liquid vehicle containing suspended polymers, particles or cells;
b) means for subjecting said sample, to flow and shear conditions within the chamber and along the inner surface of the cylindrical wall to up to 10,000 sec⁻¹, and
c) means for exposing said sample, under said flow and shear conditions in said chamber, to at least one polymer, particle or cell parameter determining operation,
wherein said suspended polymers, particles or cells are analyzed by flow cytometry for numbers of agglomerates per unit volume, as a measure of particle aggregation, the sample being analyzed from forward scatter (FSC) and side scatter (SSC) of each particle flowing past a reporting laser light beam.

19. An apparatus according to claim 18, comprising:
a housing,
a rotatable inner cylinder in said housing,
the stationary cylindrical wall spaced apart from and circumscribing said cylinder with an annular chamber defined between said wall and said rotatable inner cylinder,
motor means for rotating said inner cylinder relative to said wall,
polymer, particle or cell parameter determining means mounted in said housing for operable communication with said annular chamber.

20. Apparatus according to claim 19, wherein said stationary cylindrical wall is defined in an outer cylinder and said outer cylinder and inner cylinder are defined in a cartridge unit, removably mounted in said housing.

21. Apparatus according to claim 19, wherein said cartridge is one of a plurality of such removably mounted cartridges in said housing; said motor means collectively rotating the carrier cylinders of said plurality of cartridges.

22. Apparatus according to claim 19, wherein said cartridges are disposable.

23. Apparatus according to claim 19, wherein the annular chambers of at least some of said plurality of cartridges are of different width from annular chambers of others of said cartridges.

24. Apparatus according to claim 20, wherein said cartridge is one of a plurality of such removably mounted cartridges in said housing, and said motor means comprises individually programmable motors for each cartridge of said plurality.

25. Apparatus according to claim 20, wherein said cartridge is one of a plurality of such removably mounted cartridges in said housing, and said motor means is adapted to rotate the inner cylinders of said plurality of cartridges individually and independently, or to rotate the inner cylinders of sub-pluralities of said plurality of cartridges.

26. An apparatus according to claim 19, wherein a tube flow chamber is placed in series with the annular chamber.

27. An apparatus according of claim 26 comprising:
a housing,
a tube flow chamber in said housing,
a variable liquid flow means,
polymer, particle or cell parameter determining means mounted in said housing for operable communication with said tube flow chamber
wherein said tube flow chamber has a diameter between 0.3 to 2.0 mm,
and a restriction of diameter 0.05 to 0.2 mm, and
is selected from the group consisting of a single pass, a flow loop and a flow coil, and is removable and disposable.

28. (canceled)

29. (canceled)

30. (canceled)

31. (canceled)

32. A method according to claim 2, wherein the sample is whole blood.
